(19) United States
(12) Patent Application Publication
(10) Pub. No.: US 2024/0285685 A1
(43) Pub. Date: Aug. 29, 2024

(54) GENETICALLY MODIFIED NK CELLS AND USES THEREOF

(71) Applicant: WUXI BIOLOGICS IRELAND LIMITED, Dundalk (IE)

(72) Inventors: Yong Zheng, Shanghai (CN); Qiong Wu, Shanghai (CN); Ruixiu Cao, Shanghai (CN); Jijie Gu, Shanghai (CN)

(21) Appl. No.: 18/562,547

(22) PCT Filed: May 19, 2022
(86) PCT No.: PCT/CN2022/093747

§ 371 Date: Nov. 20, 2023

(30) Foreign Application Priority Data
May 20, 2021 (WO) PCT/CN2021/094830

(51) Int. Cl. A61K 35/17; A61P 35/00; C07K 14/725; C12N 5/0783; C12N 5/10; C12N 9/22; C12N 15/113

(52) U.S. Cl. CPC A61K 35/17; A61P 35/00; C07K 14/7051; C12N 5/0646; C12N 5/10; C12N 9/22; C12N 15/113; C12N 2310/20; C12N 2510/00

(57) ABSTRACT

Provided are genetically modified NK cells and uses thereof. The modified NK cells can be used in adoptive cellular therapy of CAR-NK cells for cancers.

### TECHNICAL FIELD

The present disclosure concerns the field of genetic engineering and immunotherapy. It inter alia pertains to genetically modified NK cells and the uses thereof in disease treatments, such as in adoptive cellular therapy and/or CAR-NK therapy of cancers.

### BACKGROUND

Natural killer (NK) cells, which make up to 15% of human peripheral blood mononuclear cells (PBMCs), are cytotoxic lymphocytes that play important roles in defense against tumor and viral infections and are now known to be an integral part of the link between the adaptive and innate immune systems.

The activity of NK cells is regulated by a repertoire of co-stimulatory (e.g. NKG2D, CD226) and co-inhibitory surface receptors (e.g. PD-1, TIGIT, CD96, TIM-3, LAG-3, NKG2A) that recognize their respective ligands on target cells or antigen-presenting cells. The integration of both co-stimulatory and co-inhibitory signals determines the responsiveness of NK cells.

TIGIT (T cell immunoreceptor with immunoglobulin and ITIM domains), a transmembrane glycoprotein receptor expressed on NK and T cells, is an immune checkpoint molecule that inhibits the activation of T cells and NK cells. It contains an IgV domain, a transmembrane domain, and an immunoreceptor tyrosine-based inhibitory motif (ITIM). CD96, a member of the same immunoglobulin superfamily, has a similar inhibitory role but with lower binding affinity for the ligand CD155 as compared to TIGIT. CD155 (mainly) and CD112 serve as ligands for TIGIT and CD96 to bind in order to inhibit T and NK cell-mediated immunity. CD155 (Poliovirus Entry Receptor, PVR) is barely expressed in normal human tissues, but highly expressed in various tumor cell lines and primary malignancies. Preclinical and clinical evidences have proved that blockade of TIGIT with monoclonal antibodies augment the antitumor and antiviral activity of NK cells and T cells.

NKG2A (NK Group 2 member A) is a NK cell receptor of the NKG2 family, a type II membrane receptor that forms a heterodimer with CD94. It dimerizes with CD94 to form an inhibitory receptor that is related to C-type lectins and recognizes HLA-E. These inhibitory receptors interact with MHC I ligands on target cells, leading to complete inhibition of cell granule polarization and the prevention of cytotoxic granule release. NKG2A contains two ITIMs in its cytoplasmic tail. These ITIMs are phosphorylated following ligation of the ITIM-bearing receptor and facilitate this leads to the recruitment of tyrosine phosphatases, such as SH2 domain-containing phosphatase (SHP)-1 and SHP-2. The recruitment of SHP-1 by the ITIM-bearing receptors seems to inhibit the initiation of signaling in that it blocks most downstream signals in NK cells. Tumor cells of hematological and solid tumors have shown upregulation of HLA-E expression. In various cancers, poor prognosis has been associated with HLA-E upregulation. Blocking of the CD94/NKG2A receptor with antibodies could be used as a therapeutic strategy.

CISH (Cytokine-Inducible SH2-containing protein), a critical negative intracellular immune checkpoint in NK cells, is a member of the intracellular suppressors of cytokine signaling (SOCS) family, which is important regulator of cytokine and growth factor signaling pathways. Similar to the other members, CISH presents a central SH2-domain, which is able to interact with phosphotyrosine residues and a SOX box motif that recruits the ubiquitin-transferase system, directing them to proteasomal degradation. CISH is rapidly induced in response to IL-15, and NK cells with deletion of CISH are more sensitive to IL-15, characterized by enhanced proliferation, cytokine production and cytotoxicity to tumors.

Genetic modification is showing promise for redirecting the function of various cell types including T cells, dendritic cells and NK cells. Much work has been done particularly on genetically redirecting T cells against a range of tumor antigens. However, difficulties in gene-modifying primary NK cells have caused this field to lag somewhat behind that of T cells. Several studies have modified NK cells with cytokine transgenes (such as IL-2, IL-12 or IL-15 transgenes) in order to enhance NK cell function by providing necessary cytokines directly to the cell. However, the majority of studies describe the redirection of NK cell specificity through chimeric receptors.

There is still a great need for NK cells with long-term persistence during preparation and use and having enhanced antitumor effects and reduced side effects.

### SUMMARY OF THE INVENTION

Disclosed herein are genetically modified NK cells and the uses thereof in disease treatments, such as in adoptive cellular therapy of cancers.

According to a first aspect, disclosed herein is an isolated genetically modified NK cell, wherein the NK cell is modified to impair the functional expression of one or more of TIGIT, NKG2A and CISH. According to some embodiments, the NK cell may further comprise a chimeric antigen receptor (CAR).

In some embodiments, an isolated modified NK cell is provided, wherein the NK cell is modified to impair the functional expression of at least two of TIGIT, NKG2A and CISH.

In some embodiments, an isolated modified NK cell is provided, wherein the NK cell is modified to impair the functional expression of one or more of TIGIT, NKG2A and CISH, and wherein the NK cell further comprises a chimeric antigen receptor (CAR).

According a second aspect, disclosed herein is a cell population or a cell culture comprising the modified NK cells of the present disclosure.

According a third aspect, disclosed herein is a product comprising the modified NK cell, the cell population or the cell culture of the present disclosure. In some embodiments, the product is a medicament, a pharmaceutical composition or a kit.

According a fourth aspect, disclosed herein is method for preparing the modified NK cell of the present disclosure.

According a fifth aspect, disclosed herein is use of the modified NK cell, the cell population, the cell culture of the present disclosure in the preparation of a product for treating diseases.

According to a sixth aspect, disclosed herein is a method for treating diseases in a subject in need thereof comprising administering an effective amount of the modified NK cell, the cell population, the cell culture or the product of the present disclosure.

According to a seventh aspect, disclosed herein is the modified NK cell, the cell population, the cell culture or the product of the present disclosure for use in treating diseases.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows the purity of the expanded NK cells.

FIG. 2 shows TIGIT knockout (KO) efficiency by CRISPR/Cas9 in NK cells tested by flow cytometry.

FIGS. 3A-3B show NKG2A knockout efficiency by CRISPR/Cas9 in NK cells tested by flow cytometry.

FIG. 4 shows TIGIT and NKG2A dual knockout efficiency by CRISPR/Cas9 in NK cells tested by flow cytometry.

FIG. 5 shows CISH knockout efficiency by CRISPR/Cas9 in NK cells tested by Western blot.

FIG. 6 shows CD155 and HLA-E expression on HT1080 tumor cells. “Negative” refers to negative control of cells stained with fluorescent labeled control antibody.

FIG. 7 shows the results of cytotoxicity assay of genetically modified NK cells on HT1080-ZsGreen target cells.

FIGS. 8A-8B show the tumor growth inhibition in A549 tumor bearing mouse after modified NK treatment. Statistic data was analyzed by Two-way ANOVA. *p<0.05; **p<0.01.

FIGS. 9A-9B show the expression of anti-CD19 CAR in CD19 CAR-NK and mCD19 CAR-NK.

FIGS. 10A-10B show the knockout efficiency of TIGIT in mCD19 CAR-NK by flow cytometry.

FIGS. 11A-11B show the knockout efficiency of CISH in mCD19 CAR-NK by Western blot.

FIGS. 12A-12B show the results of cytotoxicity assay of mCD19 CAR-NK cells on Raji-luc target cells. Statistic data was analyzed by Two-way ANOVA. *p<0.05; **p<0.01; ****p<0.0001.

FIGS. 13A-13B show the results of serial killing assay of mCD19 CAR-NK cells on Raji-luc target cells. Statistic data was analyzed by Two-way ANOVA. ***p<0.001.

FIGS. 14A-14B show cytokine release of IFN-γ in cytotoxicity assay. Statistic data was analyzed by Two-way ANOVA. *p<0.05; **p<0.01; ***p<0.001; ****p<0.0001.

FIGS. 15A-15B show cytokine release of IFN-γ in serial killing assay. Statistic data was analyzed by Two-way ANOVA. *p<0.05; **p<0.01; ***p<0.001; ****p<0.0001.

### DETAILED DESCRIPTION OF THE DISCLOSURE

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

The present disclosure is inter alia based on the unexpected finding that impairing the functional expression of TIGIT, NKG2A and/or CISH in an NK cell can remarkably improve the cytotoxicity of the genetically modified NK cell and prolong the life span of the NK cell in vitro or in vivo. Based on this finding, the disclosure provides a genetically modified NK cell and a method for the preparation thereof wherein at least one of TIGIT, NKG2A and/or CISH of the NK cell is impaired, and wherein the NK cell may further comprise or conjugated to a chimeric antigen receptor. Further provided herein is cell population, cell culture or a product, comprising the NK cell of the present disclosure and the use thereof in the treatment of disease, such as cancer, autoimmune disease, infectious disease, transplant rejection and other age-related disease.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The use of “or” means “and/or” unless stated otherwise.

As used herein, unless otherwise noted, the term “comprise”, “include” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that other elements may be present.

The term “isolated” refers to a material that is substantially or essentially free from components that normally accompany it in its native state. The material can be a cell or a macromolecule such as a protein or nucleic acid. For example, an “isolated cell,” as used herein, refers to a cell, which has been purified from the cells in a naturally-occurring state.

The term “NK cell” or “natural killer cell” refers to a type of cytotoxic lymphocyte critical to the innate immune system. NK cells mediate anti-tumor and anti-viral responses, and therefore possess promising clinical utilization. The NK cell of the present disclosure may be derived from blood (such as autologous or allogenic PBMCs), NK cell lines (such as NK-92, NKG, YT, NK-YS, HANK-1, YTS, NKL and so on), or differentiated stem cells (such as iPSC).

TIGIT, NKG2A and/or CISH

As used herein, the term “TIGIT” or “TIGIT gene” refers to a nucleotide molecule encoding T cell immunoreceptor with immunoglobulin and ITIM domains (TIGIT), an immune checkpoint molecule that inhibits the activation of T cells and NK cells.

The TIGIT gene is a gene that encodes a TIGIT polypeptide, for example a TIGIT polypeptide having a sequence set forth in SEQ ID NO: 25, or a TIGIT polypeptide sharing a high identity (for example, at least 80%, 85%, 90%, 91%, 92%, 93%, 94%, 95%, 96%, 97%, 98%, 99%, 99.5%, 99.8% identity) to the afore-mentioned TIGIT polypeptide or any TIGIT polypeptide known in the art and having the same immune checkpoint function.

For example, the TIGIT gene can be but not limited to a nucleic acid molecule having a sequence set forth in SEQ ID NO: 26; a TIGIT polypeptide encoding sequence sharing a high identity (for example, at least 80%, 85%, 90%, 91%, 92%, 93%, 94%, 95%, 96%, 97%, 98%, 99%, 99.5%, 99.8% identity) to the afore-mentioned TIGIT gene or any TIGIT gene known in the art and having the same function of coding and expressing a functional TIGIT polypeptide.

As used herein, the term “NKG2A” or “NKG2A gene” refers to a nucleotide molecule encoding NK Group 2 member A (NKG2A), a type II membrane receptor that forms a heterodimer with CD94 and interacts with HLA-E to inhibit of cell granule polarization and prevent cytotoxic granule release.

The NKG2A gene is a gene that encodes a NKG2A polypeptide, for example a TIGIT polypeptide having a sequence set forth in SEQ ID NO: 27, or a NKG2A polypeptide sharing a high identity (for example, at least 80%, 85%, 90%, 91%, 92%, 93%, 94%, 95%, 96%, 97%, 98%, 99%, 99.5%, 99.8% identity) to the afore-mentioned NKG2A polypeptide or any NKG2A polypeptide known in the art and having the same immune checkpoint function.

For example, the NKG2A gene can be but not limited to a nucleic acid molecule having a sequence set forth in SEQ ID NO: 28; a NKG2A polypeptide encoding sequence sharing a high identity (for example, at least 80%, 85%, 90%, 91%, 92%, 93%, 94%, 95%, 96%, 97%, 98%, 99%, 99.5%, 99.8% identity) to the afore-mentioned NKG2A gene or any NKG2A gene known in the art and having the same function of coding and expressing a functional NKG2A polypeptide.

As used herein, the term “CISH” or “CISH gene” refers to a nucleotide molecule encoding Cytokine-Inducible SH2-containing protein (CISH), a critical negative intracellular immune checkpoint in NK cells and a member of the intracellular suppressors of cytokine signaling (SOCS) family, which is important regulator of cytokine and growth factor signaling pathways.

The CISH gene is a gene that encodes a CISH polypeptide, for example a TIGIT polypeptide having a sequence set forth in SEQ ID NO: 29, or a CISH polypeptide sharing a high identity (for example, at least 80%, 85%, 90%, 91%, 92%, 93%, 94%, 95%, 96%, 97%, 98%, 99%, 99.5%, 99.8% identity) to the afore-mentioned CISH polypeptide or any CISH polypeptide known in the art and having the same immune checkpoint function.

For example, the CISH gene can be but not limited to a nucleic acid molecule having a sequence set forth in SEQ ID NO: 30; a CISH polypeptide encoding sequence sharing a high identity (for example, at least 80%, 85%, 90%, 91%, 92%, 93%, 94%, 95%, 96%, 97%, 98%, 99%, 99.5%, 99.8% identity) to the afore-mentioned CISH gene or any CISH gene known in the art and having the same function of coding and expressing a functional CISH polypeptide.

The term “impairing/inhibiting expression” refers to inhibiting or reducing or eliminating the expression of a gene or a protein. To inhibit or reduce or eliminate the expression of a gene (i.e., a gene encoding TIGIT, NKG2A or CISH), the sequence and/or structure of the gene may be modified such that the gene would not be transcribed (for DNA) or translated (for RNA), or would not be transcribe or translated to produce a functional protein (e.g., a transcription factor).

Various methods for inhibiting or reducing or eliminate expression of a gene are described herein or are known in the art. Some methods may introduce nucleic acid substitutions, additions, and/or deletions into the wild-type gene. Some methods may also introduce single or double strand breaks into the gene. To inhibit or reduce or eliminate the expression of a protein, one may inhibit or reduce or eliminate the expression of the gene or polynucleotide encoding the protein, as described above.

As used herein, the term “impaired” or “inhibited” expression refers to a decrease by at least 10% as compared to a reference control level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample).

As used herein, the term “inactivated” refers to preventing expression of a polypeptide product encoded by the gene. Inactivation can occur at any stage or process of gene expression, including, but not limited to, transcription, translation, and protein expression, and inactivation can affect any gene or gene product including, but not limited to, DNA, RNA (such as mRNA) and polypeptides.

In some embodiments, the gene is inhibited or inactivated by a gene deletion. As used herein, “gene deletion” refers to removal of at least a portion of a DNA sequence from, or in proximity to, a gene. In some embodiments, the sequence subjected to gene deletion comprises an exonic sequence of a gene. In some embodiments, the sequence subjected to gene deletion comprises a promoter sequence of the gene. In some embodiments, the sequence subjected to gene deletion comprises a flanking sequence of a gene. In some embodiments, a portion of a gene sequence is removed from a gene. In some embodiments, the complete gene sequence is removed from a chromosome. In some embodiments, the host cell comprises a gene deletion as described in the any of the embodiments herein. In some embodiments, the gene is inhibited or inactivated by deletion of at least one nucleotide or nucleotide base pair in a gene sequence results in a non-functional gene product. In some embodiments, the gene is inactivated by a gene deletion, wherein deletion of at least one nucleotide to a gene sequence results in a gene product that no longer has the original gene product function or activity; or is a dysfunctional gene product.

In some embodiments, the gene is inhibited or inactivated by a gene addition or substitution, wherein addition or substitution of at least one nucleotide or nucleotide base pair into the gene sequence results in a non-functional gene product. In some embodiments, the gene is inhibited or inactivated by a gene inactivation, wherein incorporation or substitution of at least one nucleotide to the gene sequence results in a gene product that no longer has the original gene product function or activity; or is a dysfunctional gene product. In some embodiments, the gene is inhibited or inactivated by an addition or substitution, wherein incorporation or substitution of at least one nucleotide into the gene sequence results in a dysfunctional gene product. In some embodiments, the host cell comprises a gene addition or substitution as described in the any of the embodiments herein.

Methods and techniques for impairing the functional expression of a gene in a host cell include, but are not limited to, clustered, regularly interspaced, short palindromic repeats (CRISPR), transcription activator-like effector nuclease (TALEN), zinc-finger nuclease (ZFN), homologous recombination, non-homologous end-joining, and meganuclease, small interfering RNA (siRNA), small hairpin RNA (shRNA; also referred to as a short hairpin RNA).

In some embodiments, TIGIT may be impaired by a CRISPR/Cas9 system comprising gRNA selected from SEQ ID NOs: 1-6, such as SEQ ID NO: 3, 2 or 6. In some embodiments, NKG2A may be impaired by a CRISPR/Cas9 system comprising gRNA selected from SEQ ID NOs: 7-18, such as SEQ ID NO: 18, 7 or 10. In some embodiments, CISH may be impaired by a CRISPR/Cas9 system comprising gRNA selected from SEQ ID NOs: 19-24, such as SEQ ID NO: 21, 19 or 24.

In some embodiments, the dual or triple knockout may be carried out by using a CRISPR/Cas9 system comprising gRNAs for two or three of TIGIT, NKG2A and CISH, such as two or more gRNAs selected from gRNAs selected from SEQ ID NOs: 1-6, gRNA selected from SEQ ID NOs: 7-18 and gRNA selected from SEQ ID NOs: 19-24. For example, the CRISPR/Cas9 system may comprise the gRNAs of SEQ ID NO: 3, 18 and/or 21.

According to the disclosure in the present application, the impairment of one or more of TIGIT, NKG2A and/or CISH may increase the in vitro and/or in vivo cell expansion; prolong the in vitro and/or in vivo cell life time; improve the in vivo cell depletion; increase the cytotoxicity of the NK cell to target cell; and/or regulate the secretion of cytokines, interleukins and/or growth factors by the NK cell.

### Chimeric Antigen Receptor (CAR)

The modified NK cells of the present application may further comprise engineered antigen receptors, such as chimeric antigen receptors (CARs), including activating or stimulatory CARs, co-stimulatory CARs (see WO2014/055668), and/or inhibitory CARs (iCARs, see Fedorov et al., Sci. Transl. Medicine, 5(215) (December 2013).

The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a co-stimulatory receptor, and/or a signal through a co-stimulatory receptor alone.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In some embodiments, the CAR comprises an antibody heavy chain domain that specifically binds the antigen, such as a cancer marker or cell surface antigen of a cell or disease to be targeted, such as a tumor cell or a cancer cell, such as any of the target antigens described herein or known in the art.

In some embodiments, the targets of the CAR include but not limited to BCMA, CD19, CD20, CD22, PSMA, ACE2, CD7, CSI, EGFR/EGFRVIII, ErBb2/HER2, CD3, CD138, and NKG2D.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs.

In some embodiments, the CAR contains an antibody or an antigen-binding fragment (e.g. scFv) that specifically recognizes an antigen, such as an intact antigen, expressed on the surface of a cell. In some embodiments, the CAR includes an anti-BCMA VHH.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD8, CD28, CD3 epsilon, CD45, CD4, CD5, CD9, CD16, CD22, CD33, CD37, CD64, CD80, CD86, CD 134, CD137, CD154. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiment, the CAR includes CD8 hinge and transmembrane region.

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The CAR generally includes at least one intracellular signaling component or components. In some embodiments, the CAR includes an intracellular component of the TCR complex, such as a TCR CD3⁺ chain that mediates T-cell activation and cytotoxicity, e.g., CD3 zeta chain. Thus, in some aspects, the antigen binding molecule is linked to one or more cell signaling modules. In some embodiments, cell signaling modules include CD3 transmembrane domain, CD3 intracellular signaling domains, and/or other CD transmembrane domains. In some embodiments, the CAR further includes a portion of one or more additional molecules such as Fc receptor γ, CD8, CD4, CD25, or CD16. For example, in some aspects, the CAR includes a chimeric molecule between CD3-zeta (CD3-2) or Fc receptor γ and CD8, CD4, CD25 or CD16.

In some embodiments, upon ligation of the CAR, the cytoplasmic domain or intracellular signaling domain of the CAR activates at least one of the normal effector functions or responses of the NK cell

In some embodiments, the CAR includes a signaling domain and/or transmembrane portion of a co-stimulatory receptor, such as CD28, 4-1BB, OX40, DAP10, and ICOS. In some aspects, the same CAR includes both the activating and co-stimulatory components; in other aspects, the activating domain is provided by one CAR whereas the co-stimulatory component is provided by another CAR recognizing another antigen.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses two or more co-stimulatory domain combined with an activation domain, e.g., primary activation domain, in the cytoplasmic portion. One example is a receptor including intracellular components of CD3-zeta, CD28, and 4-1BB.

In some embodiments, the CAR contains anti-BCMA VHH, with human CD8 hinge and transmembrane region, cytoplasmic domains 4-1BB and CD3 zeta.

In some embodiments, the CAR or other antigen receptor further includes a marker to confirm transduction or engineering of the cell to express the receptor, such as a truncated version of a cell surface receptor, such as truncated EGFR (tEGFR).

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and co-stimulatory signal, such as one including an intracellular signaling domain from a co-stimulatory receptor such as CD28 or CD137; in some aspects, a third generation CAR in some aspects is one that include multiple co-stimulatory domains of different co-stimulatory receptors.

In some aspects, the CAR or other antigen receptor is an inhibitory CAR (e.g. iCAR) and includes intracellular components that dampen or suppress a response, such as an immune response, such as an ITAM- and/or co-stimulatory-promoted response in the cell. Exemplary of such intracellular signaling components are those found on immune checkpoint molecules, including PD-1, CTLA4, LAG3, BTLA, OX2R, TIM-3, TIGIT, LAIR-1, PGE2 receptors, EP2/4 Adenosine receptors including A2AR. In some aspects, the engineered cell includes an inhibitory CAR including a signaling domain of or derived from such an inhibitory molecule, such that it serves to dampen the response of the cell, for example, that induced by an activating and/or co-stimulatory CAR. Such CARS are used, for example, to reduce the likelihood of off-target effects in the context in which the antigen recognized by the activating receptor, e.g., CAR, is also expressed or may also be expressed on the surface of normal cells. In some aspects, an inhibitory receptor, e.g., iCAR is introduced which recognizes a marker specific to the normal cell.

In some exemplary embodiments, the CAR is designed containing CD8 signal peptide (e.g., comprising SEQ ID NO: 31 or encoded by SEQ ID NO: 32); anti-CD19 scFv FMC63 (e.g., comprising SEQ ID NO: 33 or encoded by SEQ ID NO: 34); human CD8 hinge and transmembrane region (e.g., comprising SEQ ID NO: 35 or encoded by SEQ ID NO: 36); cytoplasmic domains 4-1BB (e.g., comprising SEQ ID NO: 37 or encoded by SEQ ID NO: 38); and/or CD3 zeta (e.g., comprising SEQ ID NO: 39 or encoded by SEQ ID NO: 40).

### Cell Population, Cell Culture, or Product

Also provided herein is a cell population, a cell culture, or a product comprising the modified NK cells as disclosed herein.

In some embodiments of the present disclosure, at least 50%, 50%, 60%, 65%, 70%, 75%, 80%, 85%, 90%, 91%, 92%, 93%, 94%, 95%, 96%, 97%, 98%, 99%, 99.5%, 99.8% or 100% cells in the cell population, cell culture or product are the modified NK cells of the present application. In some embodiments, the cell population, cell culture or product are free of other cells.

In some embodiments, the cell population, cell culture or product may be used for disease treatment, such as used as a pharmaceutical composition and formulation. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some embodiments, the choice of carrier is determined in part by the particular cell, binding molecule, and/or antibody, and/or by the method of administration. Accordingly, there are a variety of suitable formulations. Carriers are described, e.g., by Remington's Pharmaceutical Sciences 16th edition, Osol, A. Ed. (1980). Pharmaceutically acceptable carriers are generally nontoxic to recipients at the dosages and concentrations employed.

The formulation or composition may also contain more than one active ingredients useful for the particular indication, disease, or condition being treated with the binding molecules or cells, preferably those with activities complementary to the binding molecule or cell, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

In some embodiments, in the context of genetically engineered cells, a subject is administered the range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges, and/or such a number of cells per kilogram of body weight of the subject.

The medicament of the present disclosure may be administered using standard administration techniques, formulations, and/or devices. Provided are formulations and devices, such as syringes and vials, for storage and administration of the compositions. Administration of the cells can be autologous or heterologous. For example, immunoresponsive cells or progenitors can be obtained from one subject, and administered to the same subject or a different, compatible subject. Peripheral blood derived immunoresponsive cells or their progeny can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

### Therapeutic Methods and Uses

Also provided are therapeutic methods and uses of the engineered cells, cell population, cell culture, product of the present disclosure. The methods and uses may involve administration of the cells, or compositions containing the same, to a subject having a disease, condition, or disorder which can be treated with NK cells.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “effective amount”, in the context of administration, refers to an amount effective, at dosages/amounts and for periods of time necessary, to achieve a desired result, such as a therapeutic result. A “therapeutically effective amount” of an agent, e.g., a pharmaceutical formulation, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered.

As used herein, a “subject” is a vertebrate, e.g., a mammal, such as a human or other animal, and typically is human. In some embodiments, the subject has persistent or relapsed disease, e.g., following treatment with other therapy, including chemotherapy, radiation, and/or hematopoietic stem cell transplantation (HSCT), e.g., allogenic HSCT. In some embodiments, the subject has not relapsed but is determined to be at risk for relapse, such as at a high risk of relapse, and thus the compound or composition is administered prophylactically, e.g., to reduce the likelihood of or prevent relapse.

The diseases and disorders include cancers. Any cancer can be treated with genetically modified T cells as described herein. In some embodiments, the cancer is a hematological cancer. In some embodiments, the cancer is a carcinoma or a sarcoma. In some embodiments, the cancer is acute lymphoblastic leukemia, acute myeloid leukemia, Burkitt's lymphoma, central nervous system lymphoma, chronic lymphocytic leukemia, chronic myelogenous leukemia, hairy cell leukemia, chronic myeloproliferative disorders, a myelodysplastic syndrome, an adult acute myeloproliferative disorder, multiple myeloma, cutaneous T-cell lymphoma, Hodgkin lymphoma, or non-Hodgkin lymphoma. In some embodiments, the cancer is breast cancer, prostate cancer, testicular cancer, renal cell cancer, bladder cancer, liver cancer, ovarian cancer, cervical cancer, endometrial cancer, lung cancer, colorectal cancer, anal cancer, pancreatic cancer, gastric cancer, esophageal cancer, hepatocellular cancer, kidney cancer, head and neck cancer, glioblastoma, mesothelioma, melanoma, a chondrosarcoma, or a bone or soft tissue sarcoma. In some embodiments, the cancer is adrenocortical carcinoma, anal cancer, appendix cancer, astrocytoma, basal-cell carcinoma, bile duct cancer, bone tumor, brainstem glioma, brain cancer, cerebellar astrocytoma, cerebral astrocytoma, ependymoma, medulloblastoma, supratentorial primitive neuroectodermal tumors, visual pathway and hypothalamic glioma, or bronchial adenomas. In some embodiments, the cancer is desmoplastic small round cell tumor, ependymoma, epitheliod hemangioendothelioma (EHE), Ewing's sarcoma, extracranial germ cell tumor, extragonadal germ cell tumor, extrahepatic bile duct cancer, intraocular melanoma, retinoblastoma, gallbladder cancer, gastrointestinal carcinoid tumor, gastrointestinal stromal tumor (GIST), germ cell tumor, gestational trophoblastic tumor, gastric carcinoid, heart cancer, hypopharyngeal cancer, hypothalamic and visual pathway glioma, childhood, intraocular melanoma, islet cell carcinoma, Kaposi sarcoma, laryngeal cancer, lip and oral cavity cancer, liposarcoma, non-small cell lung cancer, small-cell lung cancer, macroglobulinemia, male breast cancer, malignant fibrous histiocytoma of bone, medulloblastoma, melanoma. Merkel cell cancer, mesothelioma, metastatic squamous neck cancer, mouth cancer, multiple endocrine neoplasia syndrome, mycosis fungoides, chronic, myxoma, nasal cavity and paranasal sinus cancer, nasopharyngeal carcinoma, neuroblastoma, oligodendroglioma, oral cancer, oropharyngeal cancer, osteosarcoma, ovarian epithelial cancer, ovarian germ cell tumor, ovarian low malignant potential tumor, paranasal sinus and nasal cavity cancer, parathyroid cancer, penile cancer, pharyngeal cancer, pheochromocytoma, pineal astrocytoma, pineal germinoma, pineoblastoma, supratentorial primitive neuroectodermal tumors, pituitary adenoma, plasma cell neoplasia, pleuropulmonary blastoma, primary central nervous system lymphoma, renal cell carcinoma, retinoblastoma, rhabdomyosarcoma, salivary gland cancer, uterine sarcoma, Sezary syndrome, non-melanoma skin cancer, melanoma Merkel cell skin carcinoma, small intestine cancer, squamous cell carcinoma, squamous neck cancer, throat cancer, thymoma, thyroid cancer, transitional cell cancer of the renal pelvis and ureter, trophoblastic tumor, gestational, urethral cancer, uterine cancer, vaginal cancer, vulvar cancer, Waldenström macroglobulinemia, or Wilms tumor.

Also among the diseases and conditions are autoimmune and inflammatory diseases. Exemplary diseases and conditions include multiple sclerosis, rheumatoid arthritis, and systemic lupus erythematosus (SLE).

Also among the diseases and conditions are age-related diseases other than cancer. Exemplary diseases and conditions include atherosis, diabetes, hepatofibrosis and ostarthritis.

In some embodiments, the methods include adoptive cell therapy, whereby genetically engineered cells of the present disclosure are administered to subjects. Such administration can promote activation of the cells (e.g., NK cell activation) in a targeted manner, such that the cells of the disease or disorder are targeted for destruction.

Adoptive cell therapy represents a new paradigm in cancer immunotherapy, but it can be limited by the poor persistence and function of transferred T/NK cells. Natural killer (NK) cells can be xenografted and have the potential to become off-the-shelf products, making NK cell or CAR-NK cell adoptive cellular therapies universal.

Here we use, for example, a CRISPR-Cas9 mutagenesis screening approach to demonstrate that, by targeting TIGIT, NKG2A and/or CISH, NK cells are reprogrammed to long-lived effector cells with extensive accumulation, better persistence and robust effector function in tumors; furthermore, the modified NK cells further comprising CAR produce improved anti-tumor effects. We thereby provide modified NK cells which are promising in the adoptive cell therapy of diseases, such as tumors.

The provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive NK cell therapy or CAR-NK cell therapy. In some embodiments, the cells or compositions are administered to the subject, such as a subject having or at risk for the disease or condition. In some aspects, the methods thereby treat, e.g., ameliorate one or more symptom of the disease or condition, such as by lessening tumor burden.

Methods for administration of cells for adoptive cell therapy are known and may be used in connection with the provided methods and compositions. In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive NK or CAR-NK cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

Depending on the type and severity of the disease, dosages of cells or pharmaceutical composition may include about 1 μg/kg to 15 mg/kg (e.g. 0.1 mg/kg-10 mg/kg), about 1 μg/kg to 100 mg/kg or more, about 0.05 mg/kg to about 10 mg/kg, 0.5 mg/kg, 2.0 mg/kg, 4.0 mg/kg or 10 mg/kg. Multiple doses may be administered intermittently, e.g. every week or every three weeks. An initial higher loading dose, followed by one or more lower doses may be administered.

After administering the cells to a mammal (e.g., a human), the biological activity of the engineered cell populations and/or the pharmaceutical composition can be measured by any of known methods. Parameters to assess include specific binding of engineered NK cells to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays. In certain embodiments, the biological activity of the cells also can be measured by assaying expression and/or secretion of certain cytokines. In some aspects the biological activity is measured by assessing clinical outcome, such as reduction in tumor burden or load.

In some embodiments, the cells or pharmaceutical composition are administered as part of a combination treatment, such as simultaneously with or sequentially with, in any order, another therapeutic intervention, such as another engineered cell or receptor or agent, such as a cytotoxic or therapeutic agent.

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

### EXAMPLES

### Example 1. NK Expansion In Vitro with K562 Feeder Cells

K562 cells expressing full length 4-1BBL and membrane-bound form of IL-21 (mbIL-21) were constructed by sleeping beauty transfection system (Addgene). Gene sequences encoding mbIL-21 and 4-1BBL were cloned into pSBbi-RB plasmid (Addgene-60522). The pSBbi-RB-mbIL21-4-1BBL plasmid was transduced into K562 cells by co-incubated with sleeping beauty transposase (SB 100×, CAT #Addgene-127909) at a ratio of 3:1. The engineered K562 cells were selected under blasticidin pressure for a few weeks and used as NK feeders to amply NK cells in vitro after confirmation of the mbIL-21 and 4-1BBL expression.

Fresh PBMCs from healthy donors were provided by SailyBio (Shanghai, China) and AllCells (Shanghai, China). The K562 feeder cells were pre-treated with 50 μg/mL mitomycin C (Sigma-M4287) for 1 hour at 37° C., 5% CO₂ to stop cell proliferation. PBMCs were co-cultured with inactivated K562 feeder cells at the ratio of 1:1 in RPMI-1640 medium containing 10% FBS and 200 U/mL (R&D-202-IL) human IL-2 at 37° C., 5% CO₂. The medium was replaced every 2 or 3 days.

### Example 2. Purity Determination of the Expanded NK Cells

At day 14, expanded NK cells (1×10⁵ cells/well) were co-incubated with (Biolegend-300439) and PE-anti-CD56 antibody APC-anti-CD3 (Biolegend-318305) at 4° C. for 1 hour. After washing with 1% BSA/PBS, the cells were further washed and resuspended in 1% BSA-PBS (w/v) for flow cytometry and the data were analyzed by FlowJo.

The purity of NK cell population (characterized by CD3 CD56+) is shown in FIG. 1 and listed in Table 1.

TABLE 1
The purity of expanded NK cells from 6 donors

| Donor | CD3⁻CD56⁺ population |
|---|---|
| 1 | 76.9% |
| 2 | 94.7% |
| 3 | 78.3% |
| 4 | 91.9% |
| 5 | 92.4% |
| 6 | 84.1% |

The result indicates that NK cells are successfully expanded with a high purity from fresh PBMCs.

### Example 3. Single or Double Knockout of TIGIT, NKG2A and/or CISH by CRISPR/Cas9

3.1 sgRNA Sequences

Small guide RNA (sgRNA) sequences for TIGIT, NKG2A or CISH were designed on the website CRISPOR (http://crispor.tefor.net/) and synthesized by GenScript (Nanjing, China). The sequences of the sgRNAs are listed in Table 2.

TABLE 2
sgRNA sequences for TIGIT, NKG2A and CISH

| TARGET | SEQ ID NO | SEQUENCES |
|---|---|---|
| TIGIT | 1 | CAGGCACAATAGAAACAACG |
| | 2 | ATGTCACCTCTCCTCCACCA |
| | 3 | GCTGACCGTGAACGATACAG |
| | 4 | TCGCTGACCGTGAACGATAC |
| | 5 | CACTGGGAGAATCTTCCTGG |
| | 6 | CTGGGTCACTTGTGCCGTGG |
| NKG2A | 7 | TGAACAGGAAATAACCTATG |
| | 8 | TTGAAGGTTTAATTCCGCAT |
| | 9 | GGTCTGAGTAGATTACTCCT |
| | 10 | AGATAAGACAGATAATTCCC |
| | 11 | ATGAGCTTCTCTGGAGCTGA |
| | 12 | AACAACTATCGTTACCACAG |
| | 13 | GCTCCAGAGAAGCTCATTGT |
| | 14 | GAAGCTCATTGTTGGGATCC |
| | 15 | CTCCATTTTAGCAACTGAAC |
| | 16 | AAGCTCATTGTTGGGATCCT |
| | 17 | ATCCCAACAATGAGCTTCTC |
| | 18 | AGGCAGCAACGAAAACCTAA |
| CISH | 19 | CAACCGTCTGGTGGCCGACG |
| | 20 | CAGGCACAATAGAAACAACG |
| | 21 | ATGTCACCTCTCCTCCACCA |
| | 22 | GCTGACCGTGAACGATACAG |
| | 23 | TCGCTGACCGTGAACGATAC |
| | 24 | CACTGGGAGAATCTTCCTGG |

### 3.2 TIGIT, NKG2A and CISH Knockout by CRISPR/Cas9

CRISPR-Cas9 ribonucleoprotein (RNP) complex was delivered to expanded NK cells by 4D-Nucleofector™ System (4D-Nucleofector Core Unit, Lonza).

Medium (RPMI-1640 containing 10% FBS and 200 U/mL human IL-2) was pre-warmed in cell culture plate at 37° C. for 30 min, and NK cells were harvested for nucleofection. RNP (ribonucleoprotein) complex of Cas9 (100 pmol, Invitrogen-A36498) and sgRNA (200 pmol) were mixed and incubated at room temperature for 20 minutes. 1×10° expanded NK cells per reaction were mixed with RNP complex comprising sgRNA gently in 100 μL P3 primary nucleofection solution (Lonza-V4XP-3024) at room temperature, and the mixture was then transferred into Nucleocuvette vessels. The vessels were inserted into Lonza 4D-Nucleofector, and nucleofection was carried out with the Program CM-137. Pre-warmed medium was immediately added into each cassette well after the vessels were removed. The medium/cell/RNP mixture was pipetted into pre-warmed RPMI-1640 medium in 6-well-plate and incubated at 37° C., 5% CO₂. At day 3, knockout efficiency was subsequently assayed by flow cytometry or Western blot.

Dual-knockout (DKO) was performed with sgRNA-3 (for TIGIT), sgRNA-18 (for NKG2A) and sgRNA21 (for CISH), which had significant effects in single protein knockout.

### 3.3 Knockout Efficiency Determination by Flow Cytometry

Single and dual knockout efficiencies of cell surface proteins TIGIT and NKG2A were determined by flow cytometry.

NK cells (1×10⁵ cells/well) were incubated with APC-anti-TIGIT antibody (eBioscience-17-9500-42) at 4° C. for 1 hour. After washing by 1% BSA/PBS, the cells were washed and resuspended in 1% BSA-PBS (w/v) for flow cytometry and the data were analyzed by FlowJo.

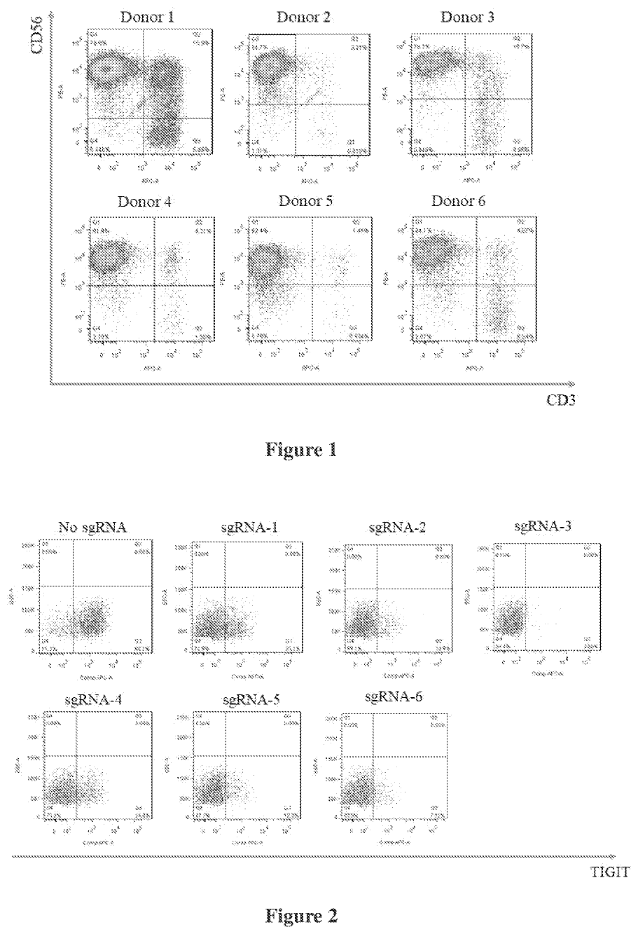

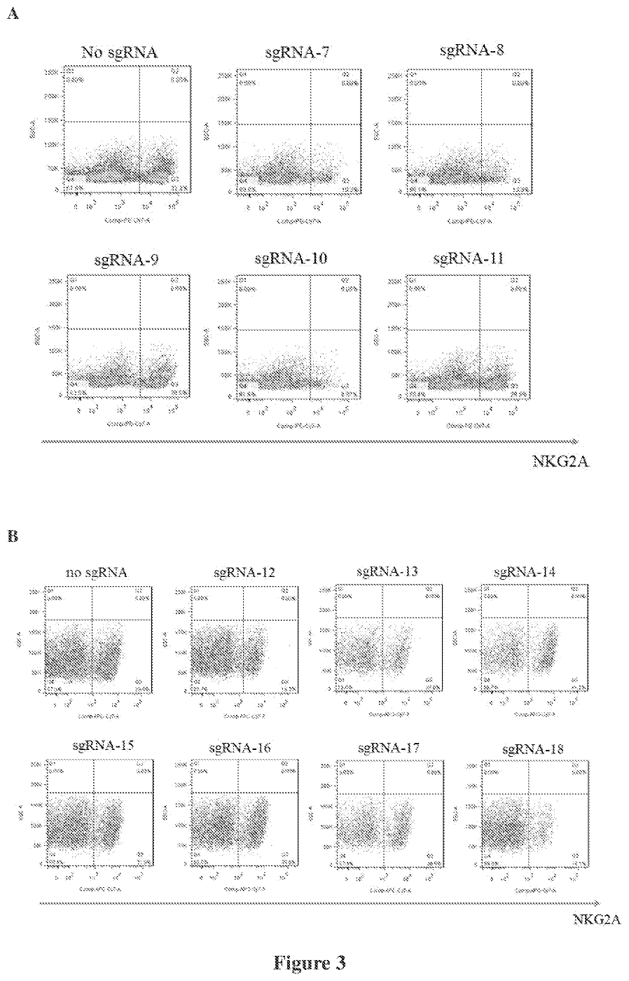

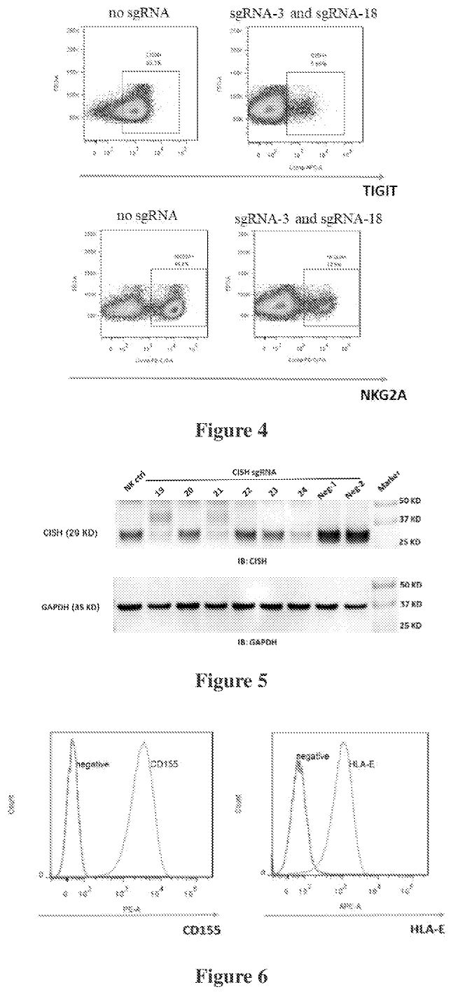

The efficiency of TIGIT, NKG2A and dual knockout are shown in FIG. 2-4 and listed in Tables 3-5, respectively.

TABLE 3
The efficiency of TIGIT knockout

| Target | SEQ No | TIGIT⁺ population |
|---|---|---|
| TIGIT | no sgRNA | 88.7% |
| | 1 | 25.1% |
| | 2 | 10.9% |
| | 3 | 2.60% |
| | 4 | 24.8% |
| | 5 | 12.3% |
| | 6 | 7.12% |

TABLE 4
The efficiency of NKG2A knockout

| Target | SEQ No | NKG2A⁺ population |
|---|---|---|
| NKG2A | no sgRNA | 32.2% |
| | 7 | 10.2% |
| | 8 | 13.9% |
| | 9 | 36.5% |
| | 10 | 8.37% |
| | 11 | 29.6% |
| | no sgRNA | 33.0% |
| | 12 | 18.3% |
| | 13 | 27.0% |
| | 14 | 41.3% |
| | 15 | 31.6% |
| | 16 | 36.5% |
| | 17 | 36.6% |
| | 18 | 10.1% |

TABLE 5
The efficiency of dual knockout of TIGIT and NKG2A

| | | TIGIT⁺ | NKG2A⁺ |
|---|---|---|---|
| Target | SEQ No | population | population |
| TIGIT and | no sgRNA | 83.3% | 46.4% |
| NKG2A | 3 and 18 | 5.66% | 12.8% |

Results indicate that at least some of the sgRNAs are effective in the knockout of TIGIT and/or NKG2A.

### 3.4 Knockout Efficiency Determination by Western Blot

Single efficiency of intracellular protein CISH was determined by Western blot.

NK cells (1×10⁷ cells) were collected, washed with ice-cold PBS and lysed in cell lysis buffer (Cell Signaling Technology-9803). Cell lysates containing equal amounts of protein were separated by SDS-polyacrylamide gel electrophoresis (PAGE) and transferred to polyvinylidene difluoride membranes. After blocking in 5% non-fat milk in Tris-buffered saline with 0.1% Tween 20, membranes were incubated at 4° C. overnight with rabbit anti-CISH antibody (Cell Signaling Technology-8731) and then were exposed HRP-goat anti-rabbit IgG (Cell Signaling Technology-7070S) for 2 h at room temperature. Immunoreactive proteins were visualized using an enhanced chemiluminescence system (ChemiDoc MF, Bio-Rad). The strips were washed in TBST and then incubated at 4° C. overnight with mouse anti-GAPDH antibody (Cell Signaling Technology-97166). Chemiluminescent signals were re-captured after secondary antibody incubation (HRP-goat anti-mouse IgG (CAT #Bethyl Laboratories-A90-231P).

The data is shown in FIG. 5. Results indicate that some of the sgRNAs were effective in CISH knockout.

### Example 4. Cytotoxicity of Modified NK Cells

Human fibrosarcoma cell line HT1080 (ECACC-no. 85111505) with endogenous CD155 expression on the cell surface and elevated HLA-E expression after IFN-γ induction, was used to assess the cytotoxicity of modified NK cells. Lentivirus expressing ZsGreen was packaged with lentivirus shuttle vector with ZsGreen synthesized in Sangon (Shanghai, China) and package plasmids PsPAX.2 (Addgene-12260) and PMD2.G (Addgene-12259). HT1080 cells transfected with lentivirus with ZsGreen were pre-seeded in a 96-well plate for 24 hours in the presence of 0.5 μg/mL IFN-γ to allow them adhere to the flat bottom of the culture plate.

The CD155 and HLA-E expression levels were determined by flow cytometry using PE-anti-CD155 antibody (eBioscience-12-1550-41) and APC-anti-HLA-E antibody (Biolegend-342606). FIG. 6 shows the high expression levels of CD155 (a ligand of TIGIT) and HLA-E (a ligand of NKG2A) in HT1080 cells.

Subsequently, modified NK cells were added to each well at the ratio of 3:10. The number of viable cells were assessed by the green fluorescence signal monitored by the IncuCyte ZOOM (Essen Bioscience) automated live cell imaging system.

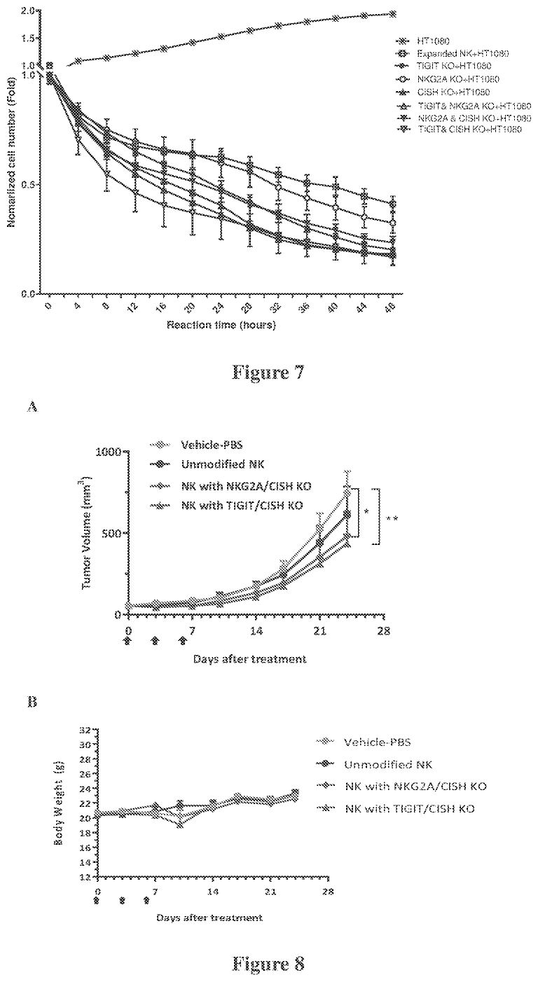

The data in FIG. 7 indicates the enhanced cytotoxicity of NK cells with TIGIT, NKG2A and/or CISH knockout at the NK/tumor cell ratio of 3:10, among which, dual knockout of TIGIT/NKG2A, TIGIT/CISH and NKG2A/CISH shows promising anti-tumor activities, with TIGIT+CISH dual knockout showing the best effect among all the tested groups.

### Example 5. In Vivo Study of Engineered NK

Six- to eight-week-old female NSG (Biocytogen, China) mice were housed and treated under specific pathogen-free conditions and were provided autoclaved food and water. All the procedures related to animal handling, care and the treatment in the study were performed following the guidance of the Association for Assessment and Accreditation of Laboratory Animal Care (AAALAC). 4×10⁶ A549 cells in 100 mL PBS were subcutaneously injected into the right flanks of NSG mice on day 0. When tumor reached 50-100 mm³, the mice were divided into four groups (Vehicle-PBS; Unmodified NK; NK with TIGIT/CISH knockout; NK with NKG2A/CISH knockout; n=6) and received 1×10⁷ NK cells intravenously at day 0, day 3 and day 6. Tumor volume and mouse weight were measured every three days. Tumor volume was measured every three days with a caliper and calculated with the following equation: V=0.5ab², where a and b are the long and short diameters of the tumor, respectively.

The results are shown in FIG. 8A (tumor growth inhibition) and FIG. 8B (body weight change), respectively.

The results in FIG. 8A show NK cells with TIGIT/CISH knockout or NKG2A/CISH knockout have superior efficacy in eliminating HT1080 tumor growth in mouse. Further, no significant differences were observed as shown in FIG. 8B, which indicated the safety of NK treatment.

### Example 6. CAR-NK and Modified CAR-NK Preparation

### 6.1 Construction of Anti-CD19 CAR Plasmid and Retroviral Vectors

The CD19 targeting CAR designed contains CD8 signal peptide (SEQ ID NO: 31 amino acid sequence; SEQ ID NO: 32 coding sequence), anti-CD19 scFv FMC63 (SEQ ID NO: 33 amino acid sequence; SEQ ID NO: 34 coding sequence), with human CD8 hinge and transmembrane region (SEQ ID NO: 35 amino acid sequence; SEQ ID NO: 36 coding sequence), cytoplasmic domains 4-1BB (SEQ ID NO: 37 amino acid sequence; SEQ ID NO: 38 coding sequence) and CD3 zeta ((SEQ ID NO: 39 amino acid sequence; SEQ ID NO: 40 coding sequence). The CAR cDNA constructs were synthesized into the multi-cloning sites of retroviral shuttle vector pMSCV-SFFV.

On day 0, 1×10 293T cells (ATCC-CRL-3216) were seeded in a 150-mm dish. Next day, CD19 CAR and vectors were co-transfected with retroviral packaging plasmids, pCMV-gag-pol and PMD2.BaEV (SEQ ID NO: 41 amino acid sequence; SEQ ID NO: 42 coding sequence) to 293T cells. On day 2, the medium of transfected 293T cells was replaced with fresh medium. On day 3, retrovirus supernatants were collected from the transfected 293T cells and were filtered with 0.45 μm polyethersulfone (PES) membrane filter. If necessary, retrovirus supernatants were concentrated by ultracentrifuge (Beckman).

Titer of retrovirus was determined by HT1080 (ECACC, no. 85111505) infection. Briefly, HT1080 cells were seeded in 96-well plate at the density of 10.000/well overnight. Serially diluted retrovirus by 5-fold with complete DMEM medium was added to HT1080 cells and mixed gently. The plate was kept in a 37° C. incubator for 72 h. The expression of CAR in HT1080 cells was characterized by fluorescence using flow cytometry. The titer of retrovirus was calculated as follows:

Titer of retrovirus (TU/mL)=(Number of cells transduced/Fluorescence positive %)/(Volume of virus).

### 6.2 Generation of Anti-CD19 CAR-NK Cells

Expanded NK cells were mixed with retroviral particles with the MOI of 4, together with polybrene (Millipore-TR-1003-G; 4 μg/mL), which had been shown to improve retroviral transduction efficiency of NK cells. Afterwards, the cells were centrifuged in a 6-well plate for 90 min (1500 rpm at 32° C.), followed by 18 hrs of incubation at 37° C. in complete RPMI 1640 medium. The transduction mixture was then removed by centrifugation and replaced with fresh complete RPMI 1640 medium, with the presence of IL-2 (200 U/mL) and IL-15 (140 U/mL).

### 6.3 Generation of Modified CAR-NK Cells

Modified anti-CD19 CAR-NK (mCD19 CAR-NK) is obtained from anti-CD19 CAR-NK on day 4 post-transfection by dual knockout of TIGIT and CISH. The electroporation process of CAR-NK with TIGIT and CISH RNP complex has been described in example 3.2. Then, mCD19 CAR-NK cells were co-cultured with mitomycin C treated K562 feeder cells (the same as the treatment in Example 1) in completed 1640 medium with the presence of IL-2 (200 U/mL) and IL-15 (140 U/mL).

### 6.4 Transduction Efficiency Determination of CD19 CAR-NK

Transduction efficiency of CD19 CAR and mCD19 CAR NK cells was evaluated at day 8 post transfection (i.e., after co-cultured with K562 feeder cells for 5 days) by flow cytometry. Briefly, CAR-NK and mCAR-NK cells were harvested and incubated with PE-labelled CD19 antigen (Acro Biosystems, CD9-HP2H3; dilution 1:100) at 4° C. for 1 hour. After washing with 1% BSA/PBS, the cells were washed and resuspended in 1% BSA-PBS for flow cytometry and the data were analyzed by FlowJo.

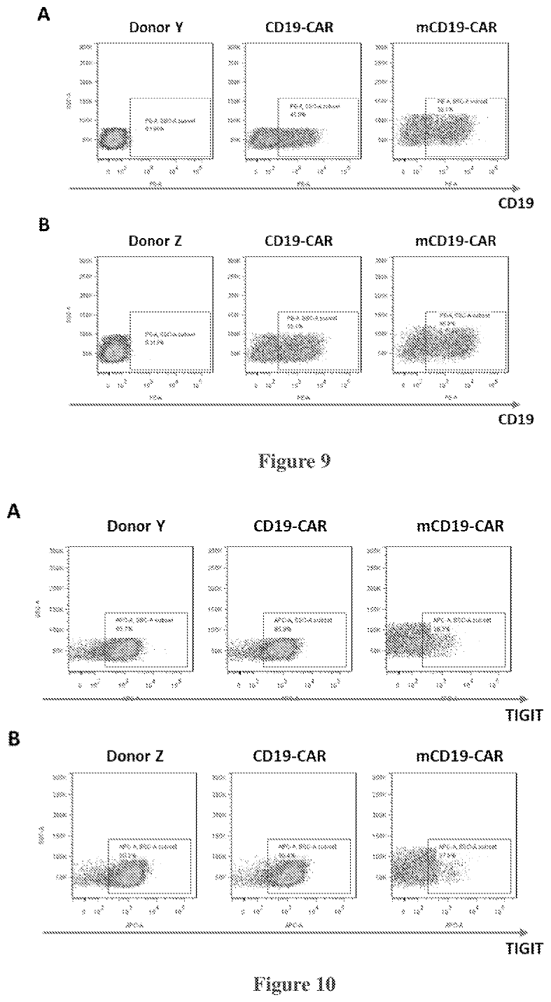

The results in FIGS. 9A and 9B show that anti-CD19 CAR-NK cells are effectively generated with high transduction efficiency.

### 6.5 Knockout Efficiency Determination by FACS and Western Blot

TIGIT and CISH knockout efficiency of mCD19 CAR NK cells was determined by flow cytometry and Western blot, respectively. The results showed in FIGS. 10A, 10B, 11A and 11B demonstrate the high knockout efficiency of TIGIT and CISH in mCD19 CAR NK cells.

### Example 7. In Vitro Characterization of CAR-NK

CAR-NK cells were characterized by the killing ability test against tumor cells and cytokine release at day 14 post transfection.

### 7.1 Cytotoxicity Assay

To generate target cells expressing luciferase, lentiviral luciferase was transduced into Raji cells. CD19 CAR-NK and mCD19 CAR-NK cells were co-cultured with 20,000 luciferase-expressing Raji (Raji-luc, ATCC-CCL86) cells at a ratio of 3:1 and 1:1 for 24 hours. One-Glo Luciferase assay reagent (CAT #Promega E6120) was added to each well. After shaking the plate for 5 min at room temperature, luminescence was detected using EnVision reader (PerkinElmer). Supernatants were collected and frozen in −80° C. for IFN-γ release. Cytotoxicity was calculated by the formula: Cytotoxicity=RLUtested group/RLU control group X 100%.

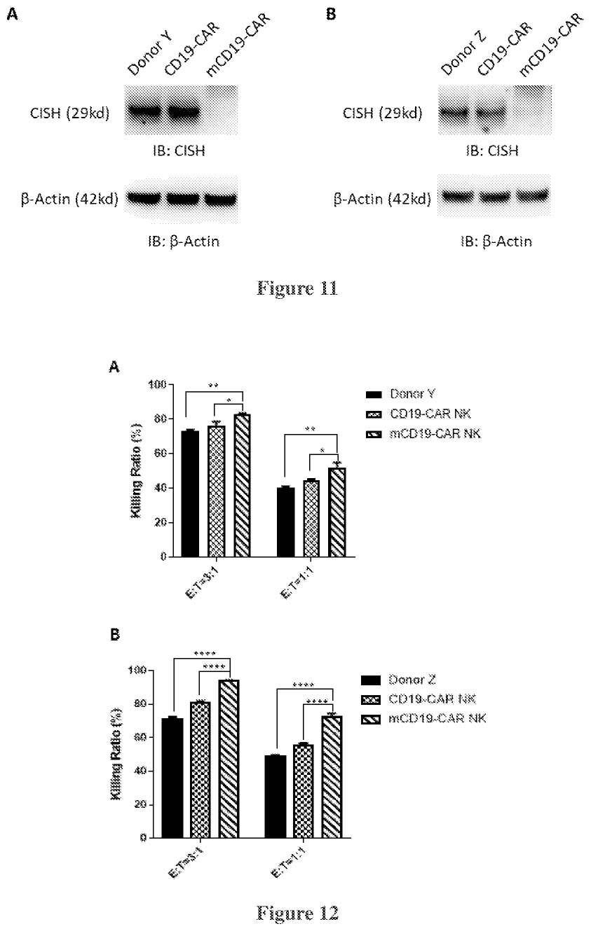

The results in FIGS. 12A and 12B show that the mCD19 CAR-NK cells have significantly increased cytotoxicity to cancer cells as compared to non-modified CD19 CAR-NK cells, indicating the modified CAR-NK cells are much more effective in cancer treatment.

### 7.2 Serial Killing Assay

Serial-killing assay was further used to explore the cytotoxicity of mCD19CAR-NK. CD19 CAR-NK and mCD19 CAR-NK cells were co-cultured with 20,000 Raji-luc cells at a ratio of 3:1 and 1:1 for 24 hours, then the same amount of new Raji-luc cells were added. 24 hours later, supernatants of 100 μL were collected and frozen in −80° C. for IFN-γ release. One-Glo Luciferase assay reagent (CAT #Promega E6120) was added to each well. After shaking the plate for 5 min at room temperature, luminescence was detected using En Vision reader (Perkin Elmer).

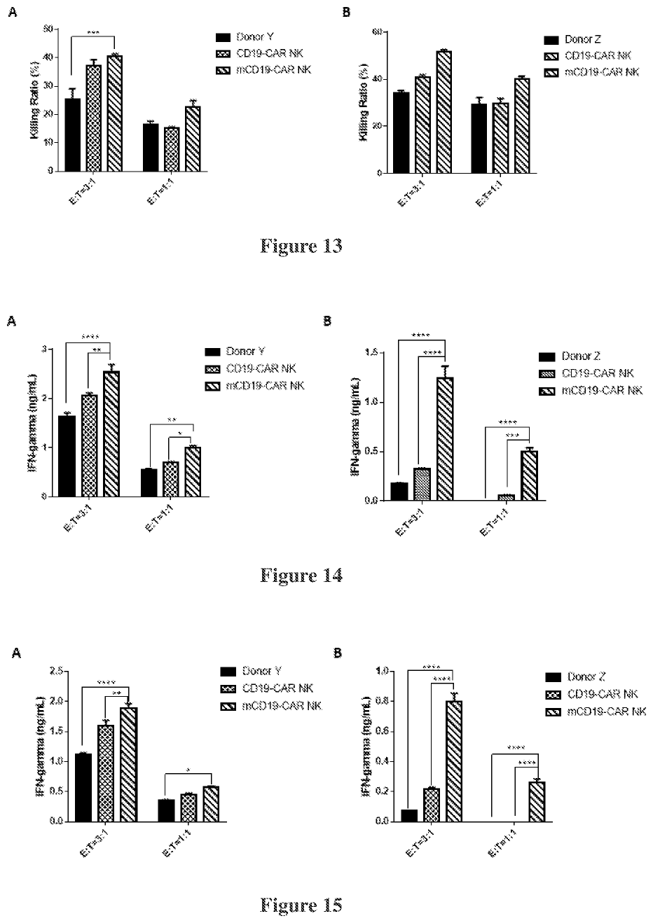

Results in FIGS. 13A and 13B show that the mCD19 CAR-NK cells have increased cytotoxicity to cancer cells in the serial killing assay, indicating the modified CAR-NK cells are potentially more effective in cancer treatment.

### 7.3 Cytokine Release Detection

The supernatant collected as described in example 7.1 and 7.2 was thawed for IFN-γ quantitation by enzyme-linked immunosorbent assay (ELISA) using matched antibody pairs.

Recombinant human IFN-γ (cat #PeproTech-300-02) was used as standards. The plates were pre-coated with capture antibody specific for human IFN-γ (cat #Pierce-M700A). After blocking, 100 μL of standards or samples were pipetted into each well and incubated for 2 hours at ambient temperature. Following removal of the unbound substances, the biotin-conjugated detecting antibody specific for IFN-γ (cat #Pierce-M701B) was added to the wells and incubated for one hour. The streptavidin conjugated Horseradish Peroxidase (HRP) (cat #Invitrogen-SNN1004) was then added to the wells for 30 min incubation at ambient temperature. The color was developed by dispensing 100 μl of TMB substrate, and then stopped by 100 μl of 2N HCl. The absorbance was read at 450 nM using a Microplate spectrophotometer.

The results in FIGS. 14A, 14B, 15A and 15B show that the release of human IFN-γ by the mCD19 CAR-NK cells is much higher than CD19 CAR-NK.

The present invention is not to be limited in scope by the embodiments disclosed herein, which are intended as single illustrations of individual aspects of the invention, and any that are functionally equivalent are within the scope of the invention. Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

APPENDIX
Sequence information

| SEQ ID | |
|---|---|
| NO: | NAME |
| 1 | TIGIT sgRNA-1 |
| 2 | TIGIT sgRNA-2 |
| 3 | TIGIT sgRNA-3 |
| 4 | TIGIT sgRNA-4 |
| 5 | TIGIT sgRNA-5 |
| 6 | TIGIT sgRNA-6 |
| 7 | NKG2A sgRNA-1 |
| 8 | NKG2A sgRNA-2 |
| 9 | NKG2A sgRNA-3 |
| 10 | NKG2A sgRNA-4 |
| 11 | NKG2A sgRNA-5 |
| 12 | NKG2A sgRNA-6 |
| 13 | NKG2A sgRNA-7 |
| 14 | NKG2A sgRNA-8 |
| 15 | NKG2A sgRNA-9 |
| 16 | NKG2A sgRNA-10 |
| 17 | NKG2A sgRNA-11 |
| 18 | NKG2A sgRNA-12 |
| 19 | CISH sgRNA-1 |
| 20 | CISH sgRNA-2 |
| 21 | CISH sgRNA-3 |
| 22 | CISH sgRNA-4 |
| 23 | CISH sgRNA-5 |
| 24 | CISH sgRNA-6 |
| 25 | TIGIT polypeptide |
| 26 | TIGIT coding sequence |
| 27 | NKG2A polypeptide |
| 28 | NKG2A coding sequence |
| 29 | CISH polypeptide |
| 30 | CISH coding sequence |
| 31 | CD8 signal peptide |
| 32 | CD8 signal peptide coding sequence |
| 33 | anti-CD19 scFv FMC63 |
| 34 | anti-CD19 scFv FMC63 coding sequence |
| 35 | human CD8 hinge and transmembrane region |
| 36 | hCD8 hinge & transmembrane region coding |
| | sequence |
| 37 | 4-1BB |
| 38 | 4-1BB coding sequence |
| 39 | CD3 zeta |
| 40 | CD3 zeta coding sequence |
| 41 | BaEV |
| 42 | BaEV coding sequence |

1. An isolated modified NK cell, wherein the NK cell is modified to impair the functional expression of one or two or three of TIGIT, NKG2A and CISH.

2. The modified NK cell of claim 1, wherein the NK cell further comprises a chimeric antigen receptor (CAR).

3. The modified NK cell of claim 1, wherein the functional expression is reduced or eliminated by gene knock-out, gene mutation, gene deletion, gene silencing or a combination of any of the foregoing.

4. The modified NK cell of claim 1, wherein the functional expression is reduced or eliminated using a CRISPR system, a TALEN system, a zinc finger nuclease (ZFN) system, a meganuclease system, an siRNA, an antisense RNA, a microRNA, a short hairpin RNA or a combination of any of the foregoing.

5. The modified NK cell of claim 1, wherein the functional expression is reduced or eliminated using a CRISPR system, and the sgRNA used is selected from SEQ ID NOs: 1-6, SEQ ID NOs: 7-18, and SEQ ID NO: 19-24.

6. The modified NK cell of claim 1, wherein the impairment of the functional expression reduces expression of the target gene(s) in the modified NK cell by at least 50, 60, 70, 80, 90, or 95% as compared to a corresponding NK cell in the absence of the impairment.

7. The modified NK cell of claim 1, wherein the NK cell is derived from the group consisting of: umbilical cord blood, peripheral blood and/or placenta of a vertebrate (e.g., human or rodent cell), and induced pluripotent stem cell (iPSC); and/or the NK cell is autologous or allogeneic.

8. The modified NK cell of claim 2, wherein the CAR comprises: (i) an antigen recognition domain, (ii) an extracellular hinge region, (iii) a transmembrane domain, and (iv) an intracellular cell signaling domain.

9. The modified NK cell of claim 8, wherein the antigen recognition domain is an antibody or antigen binding fragment thereof targets an antigen expressed on the target cells but not expressed on healthy cells, e.g., an antigen recognition domain derived from the variable regions of a monoclonal antibody (mAb) linked together as a single-chain variable fragment (scFv), or a variable domain of heavy chain of heavy chain antibody (VHH); and/or
the antigen recognition domain is a member of natural ligand/receptor pairs, e.g., a cytokine, an innate immune receptor, a TNF receptor superfamily member, a growth factor, and/or a structural protein.

10. The modified NK cell of claim 8, wherein the antigen recognition domain targets one or more antigens selected from the group consisting of: CD19, CD20, HER2, BCMA, and/or EGFR TSHR, CD19, CD123, CD22, CD30, CD171, CS-1, CLL-1, CD33, EGFRVIII, GD2, GD3, BCMA, TnAg, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-ab1, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCRI, ADRB3, PANX3, GPR20, LY6K, OR51E2, TARP, WTI, NY-ESO-1, LAGE-1a, MAGE-A1, legumain, HPV E6, E7, MAGE A1, ETV6-AML, spermprotein 17, XAGE1, Tie 2, MAD-CT-1, MADCT-2, Fos-related antigen 1, p53, p53 mutant, prostein, survivin and telomerase, PCTA-1/Galectin 8, MelanAI MART1, Ras mutant, hTERT, sarcoma translocation breakpoints, ML-IAP, ERG (TMPRSS2 ETS fusion gene), NA17, PAIX3, Androgen receptor, Cyclin B 1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRLS, and IGLL1; and, urokinase plasminogen activator receptor (uPAR).

11. The modified NK cell of claim 8, wherein the CAR comprises a CD33 endodomain (e.g. a polypeptide comprising the amino acid sequence of SEQ ID NO: 39 or encoded by the nucleotide molecule comprising SEQ ID NO: 40), a co-stimulatory signaling domain selected from CD27, CD28, 4-1BB (CD137, e.g. a polypeptide comprising the amino acid sequence of SEQ ID NO: 31 or encoded by the nucleotide molecule comprising SEQ ID NO: 32), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD1SO, IPO-3), BLAME (SLAMF8), SELPLG (CD162), LTBR, LAT, GADS, SLP-76, PAG/Cbp, NKp44, NKp30, NKp46, and NKG2D.

12. The modified NK cell of claim 8, wherein the CAR is transduced to the NK cell by a vector, e.g., a lentiviral vector or retroviral vector (such as a gamma-retroviral vector, pMSCV-SFFV); and/or by a CRISPR system.

13. (canceled)

14. The modified NK cell of claim 1 having one or more of the following characteristics as compared to a NK cell without impairing the functional expression of one or more of TIGIT, NKG2A and CISH:
(a) the functional expression of one or more of TIGIT, NKG2A and CISH is reduced or eliminated;
(b) the in vitro and/or in vivo cell expansion is increased;
(c) the in vitro and/or in vivo cell life time is prolonged;
(d) the in vivo cell depletion is improved;
(e) the cytotoxicity of the NK cell to target cell is improved; and/or
(f) the secretion of cytokines, interleukins and/or growth factors are regulated by the NK cell.

15. A method for preparing the modified NK cell of claim 1 comprising the steps of: (i) providing NK cells; (ii) modifying the NK cells to impair the functional expression of one or more of TIGIT, NKG2A and CISH; (iii) optionally modifying the NK cells to make them comprise a chimeric antigen receptor (CAR); and (iv) optionally, expanding the modified cells.

16. The method of claim 15, wherein step (ii) is carried out prior to, simultaneously with, or after step (iii); and/or step (iv) is carried out prior to or after one or more of steps (i)-(iii).

17. (canceled)

18. A method for treating diseases in a subject in need thereof comprising administering an effective amount of the modified NK cell of claim 1.

19. (canceled)

20. The method of claim 18, wherein the treatment is an adoptive cellular therapy, preferably a CAR-NK adoptive cellular therapy.

21. The method of claim 18, wherein the disease is selected from cancer, autoimmune disease, infectious disease, transplant rejection and other age-related disease.

22. The method of claim 18, wherein the disease is selected from the group consisting of carcinoma, sarcoma, melanoma, lymphoma, and leukemia; and/or a cancer selected from a cancer of hematologic system, lymphatic system, digestive system, respiratory system, reproductive system, motor system and nervous system.

23. The method of claim 18, wherein the disease is selected from the group consisting of atherosis, diabetes, hepatofibrosis and ostarthritis.


SEQUENCE LISTING (42 sequences)

SEQ ID NO: 1 - DNA, 20 bp, Artificial
  TIGIT sgRNA-1
caggcacaatagaaacaacg

SEQ ID NO: 2 - DNA, 20 bp, Artificial
  TIGIT sgRNA-2
atgtcacctctcctccacca

SEQ ID NO: 3 - DNA, 20 bp, Artificial
  TIGIT sgRNA-3
gctgaccgtgaacgatacag

SEQ ID NO: 4 - DNA, 20 bp, Artificial
  TIGIT sgRNA-4
tcgctgaccgtgaacgatac

SEQ ID NO: 5 - DNA, 20 bp, Artificial
  TIGIT sgRNA-5
cactgggagaatcttcctgg

SEQ ID NO: 6 - DNA, 20 bp, Artificial
  TIGIT sgRNA-6
ctgggtcacttgtgccgtgg

SEQ ID NO: 7 - DNA, 20 bp, Artificial
  NKG2A sgRNA-1
tgaacaggaaataacctatg

SEQ ID NO: 8 - DNA, 20 bp, Artificial
  NKG2A sgRNA-2
ttgaaggtttaattccgcat

SEQ ID NO: 9 - DNA, 20 bp, Artificial
  NKG2A sgRNA-3
ggtctgagtagattactcct

SEQ ID NO: 10 - DNA, 20 bp, Artificial
  NKG2A sgRNA-4
agataagacagataattccc

SEQ ID NO: 11 - DNA, 20 bp, Artificial
  NKG2A sgRNA-5
atgagcttctctggagctga

SEQ ID NO: 12 - DNA, 20 bp, Artificial
  NKG2A sgRNA-6
aacaactatcgttaccacag

SEQ ID NO: 13 - DNA, 20 bp, Artificial
  NKG2A sgRNA-7
gctccagagaagctcattgt

SEQ ID NO: 14 - DNA, 20 bp, Artificial
  NKG2A sgRNA-8
gaagctcattgttgggatcc

SEQ ID NO: 15 - DNA, 20 bp, Artificial
  NKG2A sgRNA-9
ctccattttagcaactgaac

SEQ ID NO: 16 - DNA, 20 bp, Artificial
  NKG2A sgRNA-10
aagctcattgttgggatcct

SEQ ID NO: 17 - DNA, 20 bp, Artificial
  NKG2A sgRNA-11
atcccaacaatgagcttctc

SEQ ID NO: 18 - DNA, 20 bp, Artificial
  NKG2A sgRNA-12
aggcagcaacgaaaacctaa

SEQ ID NO: 19 - DNA, 20 bp, Artificial
  CISH sgRNA-1
caaccgtctggtggccgacg

SEQ ID NO: 20 - DNA, 20 bp, Artificial
  CISH sgRNA-2
caggcacaatagaaacaacg

SEQ ID NO: 21 - DNA, 20 bp, Artificial
  CISH sgRNA-3
atgtcacctctcctccacca

SEQ ID NO: 22 - DNA, 20 bp, Artificial
  CISH sgRNA-4
gctgaccgtgaacgatacag

SEQ ID NO: 23 - DNA, 20 bp, Artificial
  CISH sgRNA-5
tcgctgaccgtgaacgatac

SEQ ID NO: 24 - DNA, 20 bp, Artificial
  CISH sgRNA-6
cactgggagaatcttcctgg

SEQ ID NO: 25 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 26 - DNA, 735 bp, Homo sapiens
atgcgctggtgtctcctcctgatctgggcccaggggctgaggcaggctcccctcgcctcaggaatgatgacaggcacaatagaaacaacggggaacatttctgcagagaaaggtggctctatcatcttacaatgtcacctctcctccaccacggcacaagtgacccaggtcaactgggagcagcaggaccagcttctggccatttgtaatgctgacttggggtggcacatctccccatccttcaaggatcgagtggccccaggtcccggcctgggcctcaccctccagtcgctgaccgtgaacgatacaggggagtacttctgcatctatcacacctaccctgatgggacgtacactgggagaatcttcctggaggtcctagaaagctcagtggctgagcacggtgccaggttccagattccattgcttggagccatggccgcgacgctggtggtcatctgcacagcagtcatcgtggtggtcgcgttgactagaaagaagaaagccctcagaatccattctgtggaaggtgacctcaggagaaaatcagctggacaggaggaatggagccccagtgctccctcacccccaggaagctgtgtccaggcagaagctgcacctgctgggctctgtggagagcagcggggagaggactgtgccgagctgcatgactacttcaatgtcctgagttacagaagcctgggtaactgcagcttcttcacagagactggttag

SEQ ID NO: 27 - protein, 214 aa, Homo sapiens
DNQGVIYSDLNLPPNPKRQQRKPKGNKNSILATEQEITYAELNLQKASQDFQGNDKTYHCKDLPSAPEKLIVGILGIICLILMASVVTIVVIPSRHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 28 - DNA, 648 bp, Homo sapiens
atggataaccaaggagtaatctactcagacctgaatctgcccccaaacccaaagaggcagcaacgaaaacctaaaggcaataaaaactccattttagcaactgaacaggaaataacctatgcggaattaaaccttcaaaaagcttctcaggattttcaagggaatgacaaaacctatcactgcaaagatttaccatcagctccagagaagctcattgttgggatcctgggaattatctgtcttatcttaatggcctctgtggtaacgatagttgttattccctcacgtcattgtggccattgtcctgaggagtggattacatattccaacagttgttactacattggtaaggaaagaagaacttgggaagagagtttgctggcctgtacttcgaagaactccagtctgctttctatagataatgaagaagaaatgaaatttctgtccatcatttcaccatcctcatggattggtgtgtttcgtaacagcagtcatcatccatgggtgacaatgaatggtttggctttcaaacatgagataaaagactcagataatgctgaacttaactgtgcagtgctacaagtaaatcgacttaaatcagcccagtgtggatcttcaataatatatcattgtaagcataagctttag

SEQ ID NO: 29 - protein, 258 aa, Homo sapiens
MVLCVQGPRPLLAVERTGQRPLWAPSLELPKPVMQPLPAGAFLEEVAEGTPAQTESEPKVLDPEEDLLCIAKTFSYLRESGWYWGSITASEARQHLQKMPEGTFLVRDSTHPSYLFTLSVKTTRGPTNVRIEYADSSFRLDSNCLSRPRILAFPDVVSLVQHYVASCTADTRSDSPDPAPTPALPMPKEDAPSDPALPAPPPATAVHLKLVQPFVRRSSARSLQHLCRLVINRLVADVDCLPLPRRMADYLRQYPFQL

SEQ ID NO: 30 - DNA, 777 bp, Homo sapiens
atggtcctctgcgttcagggacctcgtcctttgctggctgtggagcggactgggcagcggcccctgtgggccccgtccctggaactgcccaagccagtcatgcagcccttgcctgctggggccttcctcgaggaggtggcagagggtaccccagcccagacagagagtgagccaaaggtgctggacccagaggaggatctgctgtgcatagccaagaccttctcctaccttcgggaatctggctggtattggggttccattacggccagcgaggcccgacaacacctgcagaagatgccagaaggcacgttcttagtacgtgacagcacgcaccccagctacctgttcacgctgtcagtgaaaaccactcgtggccccaccaatgtacgcattgagtatgccgactccagcttccgtctggactccaactgcttgtccaggccacgcatcctggcctttccggatgtggtcagccttgtgcagcactatgtggcctcctgcactgctgatacccgaagcgacagccccgatcctgctcccaccccggccctgcctatgcctaaggaggatgcgcctagtgacccagcactgcctgctcctccaccagccactgctgtacacctaaaactggtgcagccctttgtacgcagaagcagtgcccgcagcctgcaacacctgtgccgccttgtcatcaaccgtctggtggccgacgtggactgcctgccactgccccggcgcatggccgactacctccgacagtaccccttccagctctga

SEQ ID NO: 31 - protein, 21 aa, Artificial
  CD8 signal peptide
MALPVTALLLPLALLLHAARP

SEQ ID NO: 32 - DNA, 63 bp, Artificial
  CD8 signal peptide coding sequence
atggccctgccagtgaccgccctgctgctgcctctggccctgctgctgcacgccgctcgtcct

SEQ ID NO: 33 - protein, 245 aa, Artificial
  anti-CD19 scFv FMC63
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 34 - DNA, 735 bp, Artificial
  anti-CD19 scFv FMC63 coding sequence
gacattcagatgacacagaccacaagcagcctgagcgcctccctgggcgacagagtgaccatcagctgcagagcctcccaagacatcagcaagtacctgaactggtatcagcagaagcccgacggcaccgtgaagctgctgatctaccacacaagcagactgcacagcggcgtgcctagcagattctccggcagcggctccggcacagactacagcctgaccatcagcaacctggagcaagaggacatcgccacctacttctgtcagcaaggcaacaccctgccctacaccttcggcgggggcaccaagctggaaatcaccggcagcacaagcgggtccggcaaacccggcagcggcgaggggagcacaaagggcgaggtgaagctgcaagagagcgggcccggcctggtggccccctcccaaagcctgagcgtgacctgcaccgtgagcggcgtgagcctgcccgactacggcgtgagctggatcagacagccccctagaaagggcctggagtggctgggcgtgatctggggcagcgagaccacctactacaacagcgccctgaagagcagactgaccatcatcaaggacaacagcaagagccaagtgttcctgaagatgaacagcctgcagaccgacgacaccgccatctactactgcgccaagcactactattacggcggcagctacgccatggactactggggccaaggcacaagcgtgaccgtgagcagc

SEQ ID NO: 35 - protein, 69 aa, Artificial
  human CD8 hinge and transmembrane region
TTTPAPRPPTPAPTIASQPLSLRPEACRPAAGGAVHTRGLDFACDIYIWAPLAGTCGVLLLSLVITLYC

SEQ ID NO: 36 - DNA, 207 bp, Artificial
  human CD8 hinge and transmembrane region coding sequence
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 37 - protein, 42 aa, Artificial
  4-1BB
KRGRKKLLYIFKQPFMRPVQTTQEEDGCSCRFPEEEEGGCEL

SEQ ID NO: 38 - DNA, 126 bp, Artificial
  4-1BB coding sequence
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 39 - protein, 112 aa, Artificial
  CD3 zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - DNA, 336 bp, Artificial
  CD3 zeta coding sequence
cgggtgaagttctccaggtctgccgacgcccccgcctacaagcagggccagaaccagctgtacaatgagctgaacctgggccggcgggaggagtatgacgtgctggataagaggcgcggccgggaccccgagatgggcggtaaacctagaagaaaaaatccacaggaaggactgtataatgaactgcagaaggataaaatggctgaggcatatagtgaaattggcatgaaaggggagaggagaagaggaaaaggacatgatggactgtatcagggactgagcacagctacaaaagatacatatgatgccctgcacatgcaggccctgccccctaga

SEQ ID NO: 41 - protein, 563 aa, Artificial
  BaEV
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 42 - DNA, 1692 bp, Artificial
  BaEV coding sequence
atgggattcacaacaaagataatcttcttatacaacctagtactggtctacgcggggtttgacgaccctcgcaaagccatagaactagtacaaaagcgatatggccgaccatgcgattgcagcggaggacaagtgtccgagcccccgtcagacagggtcagtcaagtgacttgctcaggcaagacagcttacttaatgcccgaccaaagatggaaatgtaagtcaattccaaaagacacctccccaagcgggccactccaagagtgcccctgtaattcttaccagtcctcagtacacagttcttgttatacctcataccaacaatgcagatcaggcaataagacatattatacggctactctgctaaaaacacaaactgggggcaccagtgatgtacaagtattaggatccaccaacaaacttatacaatctccctgtaatggcataaaagggcagtctatttgctggagcactacagctcctatccacgtctctgatggaggaggtccattagacaccacaagaattaaaagtgttcagagaaaactggaagaaattcataaagccctatatcctgaacttcagtatcaccctttggccatacctaaggttagagataacctcatggtcgatgcccagactttaaacattctcaatgccacttacaacttactcctaatgtccaacacgagcctagtggacgactgttggctttgtttaaaattaggtccccctactcccctcgcaatacctaacttcctattatcctacgtgactcgctcctcggataatatctcttgtttaataattcccccccttctagttcaaccgatgcagttttccaattcatcttgcctcttttccccctcctacaacagtacagaagaaatagatctaggccatgttgccttcagcaactgtacctccataaccaatgtcaccggtcccatatgcgctgtaaatggttcggtctttctctgtggcaataacatggcatacacttatctacccacgaactggacggggctttgcgtcctagcaactctcctccccgacattgacatcattcccggagatgaaccggtccccatccctgctattgatcattttatatatagacctaaacgggccatacagtttattcctttactagcagggctagggatcaccgcagccttcacaacaggagctacaggcctaggtgtctctgtgacccaatatacaaaattatctaatcagctaatttctgatgtacaaatcttatctagcaccatacaagatctgcaagatcaagtagactcattagccgaagtggttctccagaacagaagggggctagatctacttacagcagaacaaggaggaatctgtttagccctgcaagaaaaatgctgcttttatgttaacaagtcagggattgtgagagacaaaataaaaaccttacaagaagaactagaaagacgtagaaaagatctagcttccaacccactttggactgggcttcaagggctcctcccttacctcctgccctttcttggccctctacttaccctcctgctcttactcaccattgggccgtgcatttttaaccgtctaaccgcttttattaatgataagttaaacataatacacgctatggtgctaacccaacagtatcaggtgctcagaaccgatgaagaagctcaagattga